(19) United States
(12) Patent Application Publication
(10) Pub. No.: US 2011/0245806 A1
(43) Pub. Date: Oct. 6, 2011

(54) REINFORCED MULTI-LUMEN CATHETER AND METHODS FOR MAKING SAME

(75) Inventor: Ryan C. Patterson, Farmington, UT (US)

(73) Assignee: C. R. Bard, Inc., Murray Hill, NJ (US)

(21) Appl. No.: 13/078,734

(22) Filed: Apr. 1, 2011

Related U.S. Application Data
(60) Provisional application No. 61/320,617, filed on Apr. 2, 2010.

(51) Int. Cl. A61M 25/00; B29C 47/00

(52) U.S. Cl. 604/524; 264/209.1; 29/428

(57) ABSTRACT

Methods for manufacturing multi-lumen catheter tubes are disclosed. The described methods may be employed to manufacture a reinforced catheter tube suitable for use in power injectable and torqueable multi-lumen catheters, for example. In one embodiment, a method for manufacturing a multi-lumen catheter tube comprises first clamping a flexible sheet of catheter tube material between first and second mandrels. The mandrels may be D-shaped, or may include another suitable cross sectional profile. The first and second mandrels are rotated with respect to the flexible sheet so as to wrap the flexible sheet about the mandrels. Adjacent portions of the flexible sheet are joined along a longitudinal length thereof to define a closed catheter tube including first and second lumens. The first and second lumens may conform to the D-shaped profile of the respective mandrels.

### CROSS-REFERENCE TO RELATED APPLICATIONS

This application claims the benefit of U.S. Provisional Patent Application No. 61/320,617, filed Apr. 2, 2010, and entitled “Reinforced Multi-Lumen Catheter and Methods for Making Same,” which is incorporated herein by reference in its entirety.

### BRIEF SUMMARY

Briefly summarized, embodiments of the present invention are directed to multi-lumen catheter tubes and methods for manufacturing such catheter tubes. The described methods may be employed to manufacture a reinforced catheter tube suitable for use in high pressure fluid flow applications, such as power injection of fluids through a catheter into a vasculature of a patient. In addition, the catheter tubes to be described herein also exhibit desirable torque response, kink resistance, and pushability, according to one embodiment.

In one embodiment, a method for manufacturing a reinforced multi-lumen catheter tube includes providing a plurality of elongate beading elements. A catheter tube is overextruded over the beading elements such that each beading element is disposed in and defines a cross sectional profile of a lumen of the catheter tube. The catheter tube can then be reinforced, such as by a braiding applied to the outer surface thereof. An outer covering is extruded over the reinforced catheter tube to complete the assembly.

In another embodiment, a method for manufacturing a multi-lumen catheter tube comprises first clamping a flexible sheet of catheter tube material between first and second mandrels. The mandrels may be D-shaped, or may include another suitable cross sectional profile. The first and second mandrels are rotated with respect to the flexible sheet so as to wrap the flexible sheet about the mandrels. Adjacent portions of the flexible sheet are joined along a longitudinal length thereof to define a closed catheter tube including first and second lumens. The first and second lumens may conform to the D-shaped profile of the respective mandrels.

These and other features of embodiments of the present invention will become more fully apparent from the following description and appended claims, or may be learned by the practice of embodiments of the invention as set forth hereinafter.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 is a perspective view of a multi-lumen catheter manufactured using a method in accordance with one embodiment;

FIG. 2 is a perspective view of a beading element for use in manufacturing a catheter according to one embodiment;

FIG. 2A is a perspective view of a roll of the beading element in FIG. 1, showing the beading element being spooled out;

FIGS. 3A-3C show various stages of a method for manufacturing a multi-lumen catheter according to one embodiment;

FIGS. 4A-4C show various stages of a method for manufacturing a multi-lumen catheter according to one embodiment;

FIGS. 5A-5B show various stages of a method for manufacturing a multi-lumen catheter according to one embodiment;

FIGS. 6A-6E show various stages of a method for manufacturing a multi-lumen catheter according to one embodiment;

FIGS. 7-10 show possible weaving configurations for forming a catheter tube from a flexible sheet, according to one embodiment;

FIGS. 11A-11C show a possible weaving configuration for forming a catheter tube from a sheet, according to one embodiment;

FIGS. 12A-12D show various stages of a method for manufacturing a multi-lumen cannula for use with a catheter according to one embodiment;

FIGS. 13A-D show various stages of a method for manufacturing a multi-lumen catheter according to one embodiment;

FIGS. 14A-14F show various stages of a method for manufacturing a multi-lumen catheter according to one embodiment;

FIG. 15 shows a simplified view of a device for manufacturing a multi-lumen catheter according to one embodiment; and

FIG. 16 is a perspective view of a series of mandrels aligned with a sheet of material in accordance with a method of manufacturing a multi-lumen catheter according to one embodiment.

### DETAILED DESCRIPTION OF SELECTED EMBODIMENTS

Reference will now be made to figures wherein like structures will be provided with like reference designations. It is understood that the drawings are diagrammatic and schematic representations of exemplary embodiments of the present invention, and are neither limiting nor necessarily drawn to scale.

For clarity it is to be understood that the word “proximal” refers to a direction relatively closer to a clinician using the device to be described herein, while the word “distal” refers to a direction relatively further from the clinician. For example, the end of a catheter placed within the body of a patient is considered a distal end of the catheter, while the catheter end remaining outside the body is a proximal end of the catheter. Also, the words “including,” “has,” and “having,” as used herein, including the claims, shall have the same meaning as the word “comprising.”

Embodiments of the present invention are generally directed to multi-lumen catheters and methods for making such catheters. In one embodiment, the multi-lumen catheter includes reinforcing structure to enable the catheter to be employed for power injection, i.e., injection of contrast media or other fluids through the catheter at elevated flow rates and/or fluid pressures. In one embodiment, power injection includes fluid flow through the catheter at flow rates of about 5 ml per second and fluid pressures of about 300 psi, though other flow rates and pressures are also possible. To achieve such power injection, in one embodiment a power injector is operably connected to a proximal portion of the catheter. The catheter structures and catheter forming methods to be described herein present configurations for high strength catheters that can withstand such power injection and are readily manufacturable.

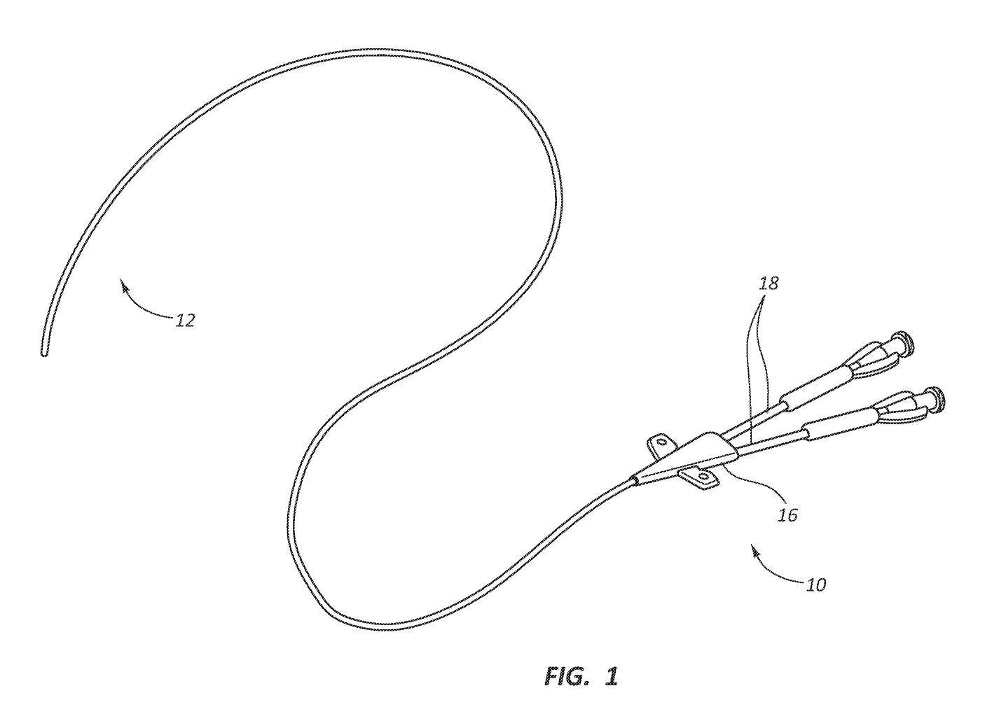

Reference is first made to FIG. 1, which shows a catheter 10 that is manufacturable by a method in accordance with one embodiment. As shown, the catheter 10 includes a catheter tube 12 defining two lumens 14, though more than two lumens can be included in other embodiments. A bifurcation 16 is included at a proximal end of the catheter tube 12 to interconnect the lumens 14 thereof to a respective one of two extension legs 18. Note that the catheter 10 shown in FIG. 1 is a peripherally inserted central catheter, or PICC, and is but one example of a catheter that can be manufactured in accordance with the principles described herein. Other catheters to establish vascular or other access to a body of a patient can also benefit from the present disclosure and thus the principles of the present disclosure should therefore not be limited to what is explicitly shown and described herein.

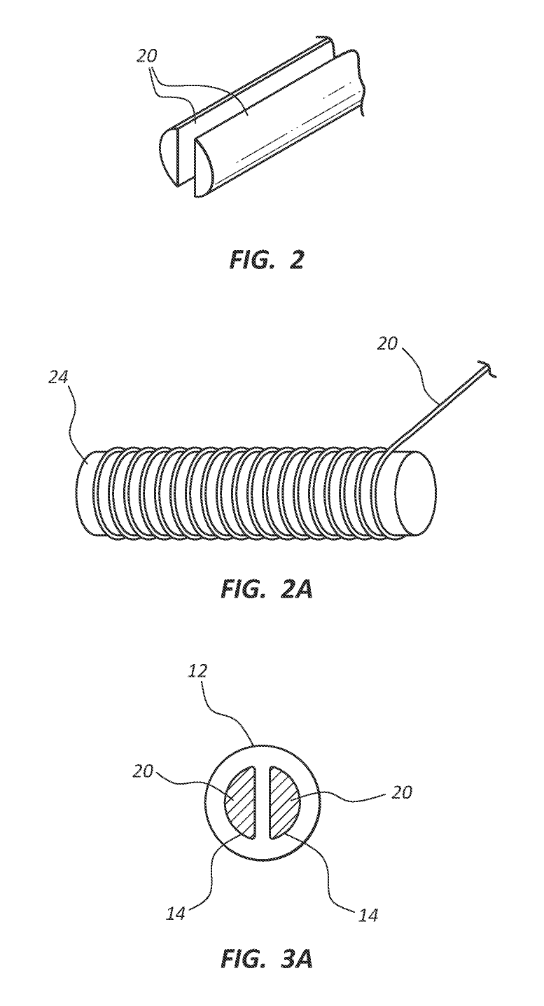

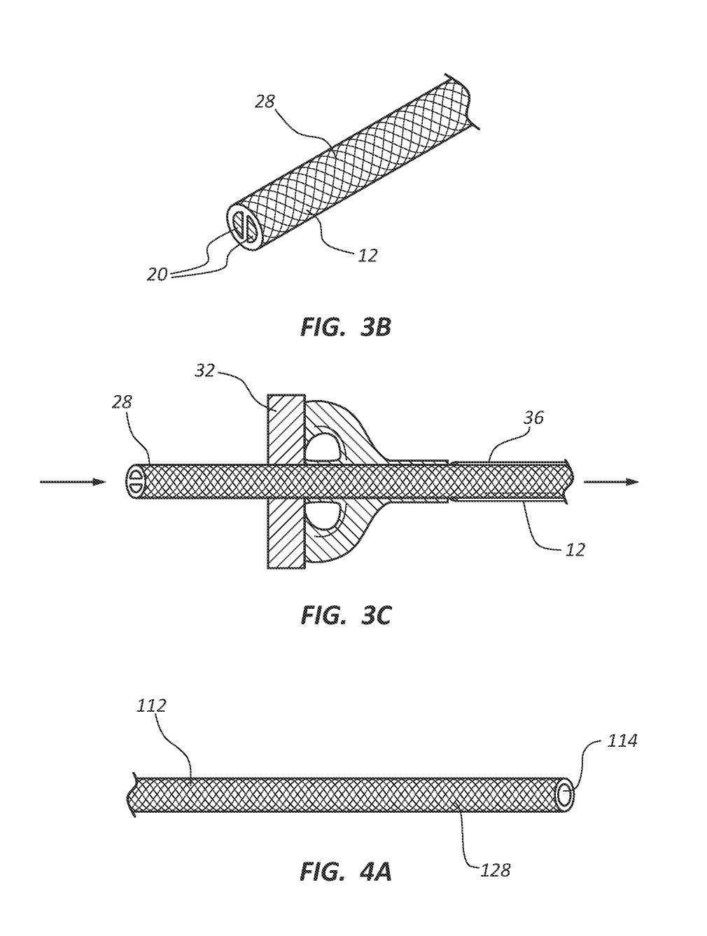

FIGS. 2-3C depict various details regarding the manufacture of a multi-lumen catheter according to one embodiment. As with the other embodiments described herein, the catheter can be manufactured as either a reinforced catheter capable of withstanding power injection, or as a non-reinforced catheter.

In particular, FIG. 2 shows a set of two elongate, D-shaped beading elements (“beading”) 20 used in the present embodiment to form a multi-lumen catheter. The beading 20 can be formed from PTFE or other suitable material via an extrusion or other suitable process, then spooled onto relatively large spools, such as the beading spool 24 shown in FIG. 2A, including dimensions of about four feet in length by about one foot in height in one embodiment. Spooling of the beading 20 in this manner can assist in maintaining straightness of the beading when used in catheter forming as described in FIGS. 3A-3C, according to one embodiment.

In greater detail and as shown in FIG. 3A, in one embodiment the two lengths of beading 20 as shown in FIG. 2 are positioned with flat sides facing each other and fed from the beading spool 24 into an extruder to form an elongate catheter tube 12 having dual lumens 14, each lumen being occupied by a corresponding beading after the extrusion process. Note that the beading 20 are formed so as to define a cross sectional profile that matches the desired cross sectional profile of the respective lumen 14 of the catheter tube manufactured here.

FIG. 3B shows that a reinforcement can be added to the extruded catheter tube 12 so as to impart to the catheter the ability to withstand relatively high fluid throughput and pressures encountered during power injection or similar procedures. In the present embodiment, the reinforcement includes a reinforcement braiding 28 that can be added to the exterior of the catheter tube 12 during or after the initial extrusion and in any one of a number of suitable ways. Other reinforcements can also be provided to the catheter tube. The braiding or other suitable reinforcement can include nylon, para-aramid synthetic fibers sold under the mark KEVLAR™, polymer fibers, etc. Again, in this and the other methods described herein, the reinforcement may be optionally omitted from the catheter tube.

As shown in FIG. 3C, once reinforced the catheter tube 12 is fed through a coextrusion head 32 to provide an outer covering 36 to the catheter tube. The beading 20 can be removed from the lumens 14 of the catheter 12 at a suitable stage in the manufacturing process, such as after the outer covering 36 is applied. The catheter tube 12 can then be further processed to result in a reinforced catheter such as that shown at 10 in FIG. 1.

Note that, though this and the other embodiments describe methods for forming dual lumen catheters, in other embodiments it is appreciated that the principles described herein can be expanded to the formation of catheters having one, three, or more lumens. It is appreciated that in this and other embodiments herein, the catheter tube can include one or more of a variety of suitable materials, including thermoplastic elastomers (e.g., HDPE, polyurethane, PEBAX™, Nylon, etc.), thermosets such as silicone, and the like.

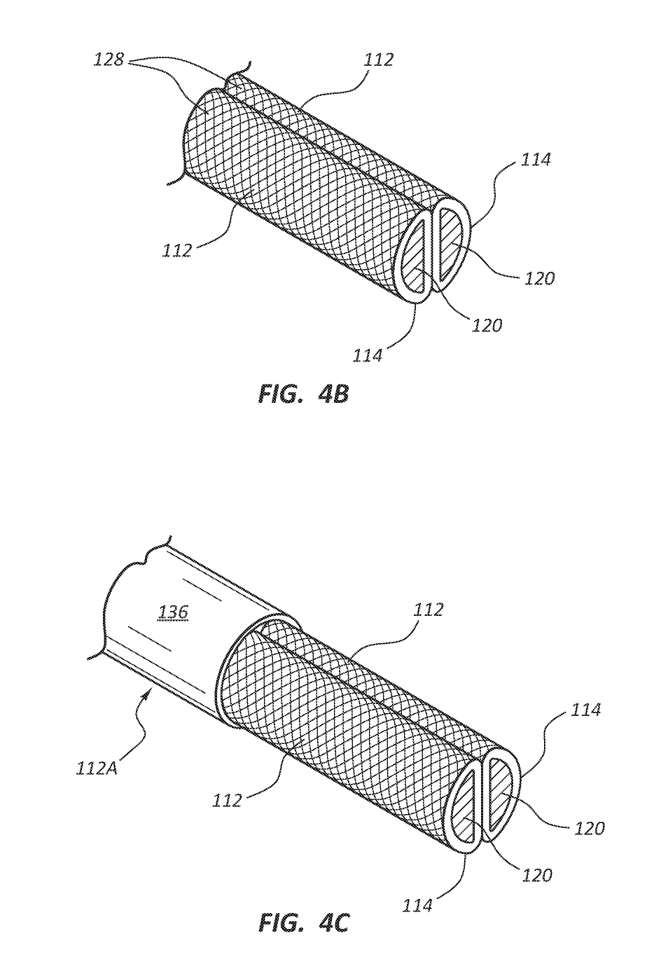

Another method for manufacturing a multi-lumen catheter is described in FIGS. 4A-4C. In FIG. 4A a catheter tube 112, defining a single lumen 114 and including a reinforcement such as braiding 128, is formed. In one embodiment, an extrusion-braiding-overextrusion procedure is employed to define the reinforced catheter tube 112 with an inner layer, an interposed braiding layer, and an outer layer. This process is repeated to produce a second reinforced catheter tube 112.

The two catheter tubes 112 are fitted to D-shaped mandrels 120, wherein the lumen 114 of each catheter tube 112 receives one of the mandrels so as to constrain the cross sectional profile of the lumen to a D-shape, as shown in FIG. 4B. Of course, other mandrels defining other cross sectional profiles can be used.

The two catheter tubes 112 can be fixtured or adhered together before an outer covering 136 is added to cover both tubes, as shown in FIG. 4C. The assembly is then subjected to a heat and pressure procedure, such as a lay-up process, to heat-set the assembly and form a composite multi-lumen catheter tube 112A. The mandrels 120 are then removed and the catheter tube further processed for inclusion in a catheter. Note that in one embodiment the catheter tubes may be braided using a continuous process before the tubes are cut and prepared for application of the outer covering.

Note that, in another embodiment, the two catheter tubes are optionally extruded over D-shaped beading to provide each tube with a D-shaped lumen, similar to that seen in FIG. 4B. Other beading elements with other non-round cross sectional shapes could also be used. The two tubes are each reinforced, then joined together by the outer covering to define a multi-lumen catheter tube. As with other embodiment described herein, more than two lumens can be included in such a catheter tube.

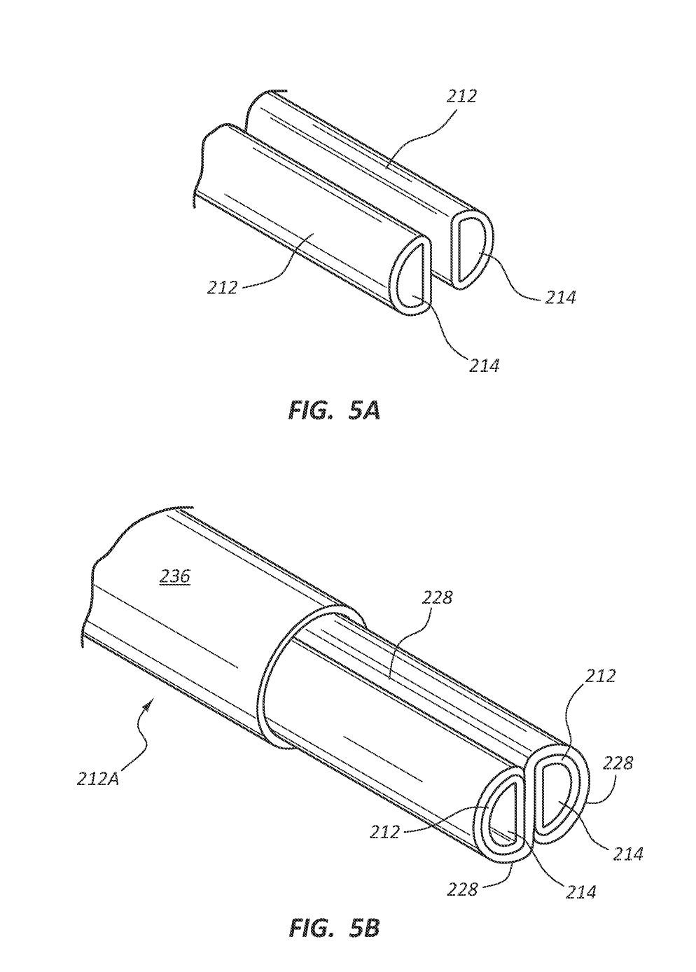

FIGS. 5A and 5B depict a catheter forming method according to yet another embodiment, wherein two catheter tubes 212 are formed to each define a lumen 214. The lumens 214 include a predetermined cross sectional profile, such as D-shaped for instance. Such a cross sectional profile can be achieved, for instance, via shaping an extruded round lumen over a D-shaped mandrel, a D-shaped extrusion over a D-shaped beading or mandrel, etc. If no reinforcement is desired, the catheter tubes 212 can be joined together, an outer covering added, and a heat and pressure process performed to form a composite multi-lumen catheter tube.

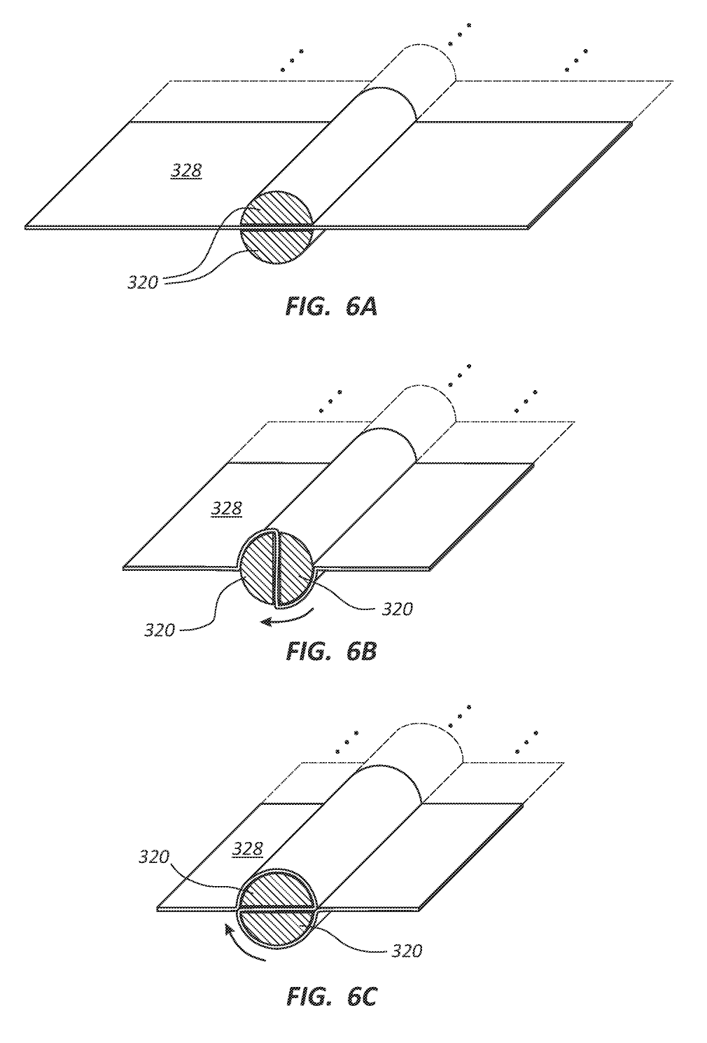

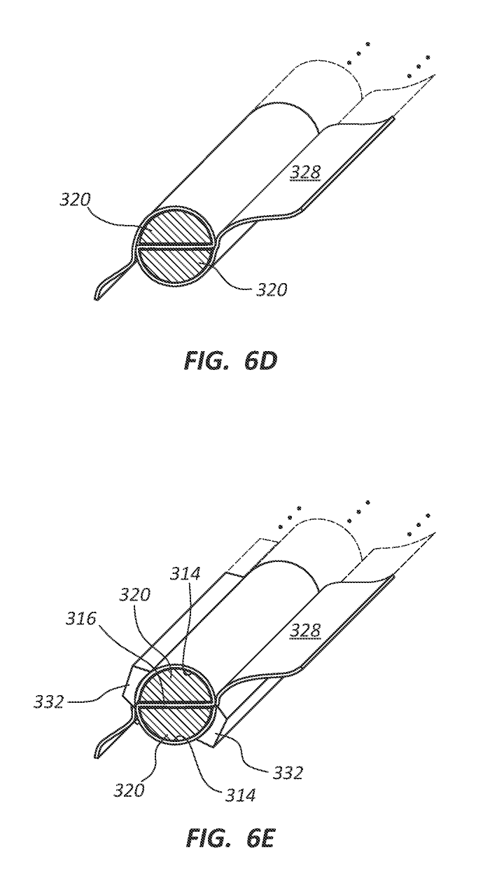

If reinforcement of the catheter tubes 212 is desired, in one embodiment a reinforcement sheet 228 is wrapped in an intertwining fashion around the catheter tubes in the manner shown in FIG. 5B, or in some other suitable wrapping configuration that provides circumferential reinforcement to each of the catheter tube lumens 214, before an outer covering is added thereto. The sheet can be a variety of lengths to accommodate wrapping about a catheter tube of similar length. Note that wrapping of the above reinforcement sheet about the catheter tubes can proceed as described below in connection with FIGS. 6A-12C, in one embodiment.

Non-limiting examples of reinforcement materials for use in the above embodiment as described in connection with FIGS. 5A and 5B and for the other reinforcement sheet embodiments herein include a fiber-impregnated thermoplastic elastomer, such as polyurethane, and a fiber-impregnated thermoset, such as silicone. The fibers in one embodiment include metal, nylon, PTFE, glass, etc. Yet other reinforcement sheet materials include woven and non-woven polymers, corrugated sheeting, stainless steel, nickel-titanium metal alloys such as nitinol or other shape memory materials, woven films, microspun nanotubes, etc.

As mentioned, after the reinforcement sheet has been suitably wrapped about the catheter tubes 212, an outer covering 236 is added thereto via a heat and pressure procedure or other suitable process to form a composite catheter 212A. Though two catheter tubes 212 are shown here, more or fewer than two catheter tubes can be included in the composite catheter, with the cross sectional profile of each tube being modified as needed for the particular application.

FIGS. 6A-6E depict yet another catheter forming method, according to one embodiment, wherein a flexible sheet of suitable catheter tube material is employed to form the catheter tube in order to take advantage of enhanced engineering or material properties that can be found in materials in sheet form. Indeed, it is appreciated that materials manufactured in sheet form can be formed suitably thin and can exhibit desirable properties useful for catheter formation. In one embodiment, for example, the sheet of catheter material can be made to vary over the length of the catheter tube, e.g., a variable durometer sheet wherein one end of the sheet has a relatively stiff durometer that differs from the other, softer end. Such a sheet, continuously or discretely variable along the length thereof, can be utilized to produce a catheter tube that is softer toward the distal tip of the tube than more proximal portions.

The sheet of catheter tube material can be formed from any suitable process, including extrusion, a solvent-based process, or a layering process, among others. In the layering process, for example, successive layers of identical or distinct materials are bonded to one another via pressure and heat, for instance, to define a highly engineered composite sheet. The sheet can include materials as described above in connection with FIGS. 5A and 5B, such as fiber-impregnated thermoplastic elastomers, fiber-impregnated thermosets, etc. Optionally, a self-adhesive layer can be included in the catheter tube material sheet to aid in assembly and bonding of the catheter tube.

As shown in FIG. 6A, in the present embodiment a flexible forming sheet 328 is interposed between two D-shaped, spaced apart and aligned mandrels 320 such that the sheet is clamped therebetween. The mandrels 320 are then rotated in unison about a common longitudinal axis while sufficient resistance is maintained on the sheet 328 so as to cause the sheet to wrap around the mandrels in a manner shown in FIGS. 6B-6D. As mentioned, in one embodiment the spacing between the mandrels is small enough to enable the mandrels to clamp and hold the sheet and prevent its slippage from between the mandrels during rotation. In another embodiment, the sheet is rotated about stationary mandrels. In yet another embodiment, the sheet is interposed between the mandrels, but not clamped thereby.

Once the mandrels have been rotated sufficient to wrap the forming sheet 328 completely around the mandrels, such as 180 degrees rotation in one embodiment, the excess sheet material, if any, is cut off and the remaining ends of the forming sheet are bonded to adjacent sheet portions (via ultrasonic, RF or other suitable welding, heat staking, use of an adhesive, shrink-down process using shrink tubing material such as FEP, etc.) along the longitudinal length of the rotated sheet to seal the catheter lumens and form a completed and closed-wall catheter tube (the tube ends remain open). In the case of joining the free ends of the forming sheet 328 via welding to form the catheter tube, sufficient heat and pressure can be used in one embodiment to produce a suitable bond.

In one embodiment a cutting fixture 332 (FIG. 6E) is run longitudinally down the length of the rotated sheet prior to bonding of the sheet ends in order to cut away the excess sheet material. The mandrels 320 are then removed and the resulting catheter tube includes the two lumens 314 and a septum 316 therebetween, each defined by corresponding portions of the forming sheet 328. In particular, the lumens 314 each include a cross sectional profile matching that of the mandrels 320 about which the forming sheet 328 rotated. It is appreciated that in one embodiment the sheet can be sized and aligned with the mandrels such that no cutting step is needed, and the ends need simply be bonded to adjacent portions of the rolled sheet.

If needed, a centerless grinding or other suitable process can be used to smooth the outer surface of the catheter. In another embodiment, an outer covering formed via a layup or overextrusion process for instance, can be applied to the catheter, if desired. Catheters including fewer or more lumens can be defined by this method, in other embodiments.

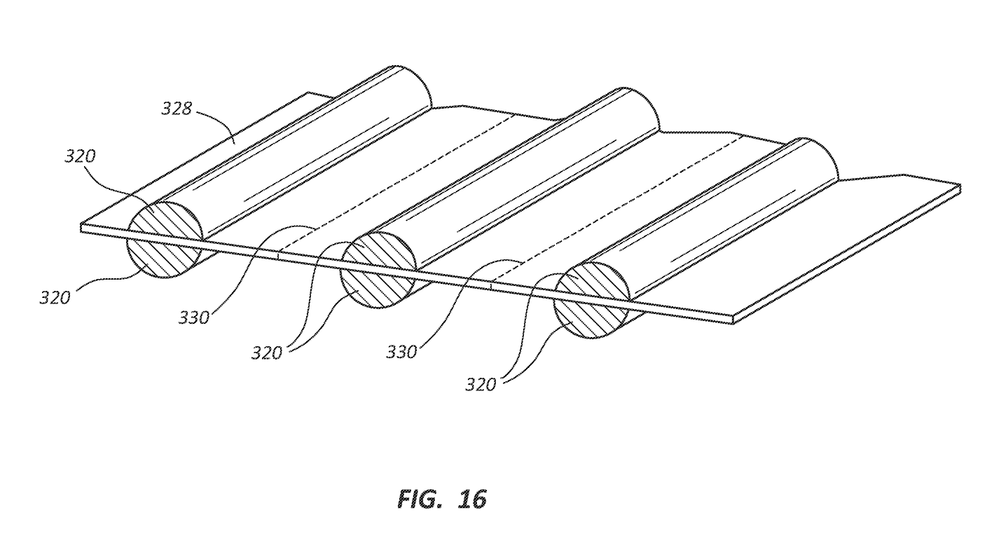

As shown in FIG. 16, the sheet 328 in one embodiment can be sized and sub-divided into suitably sized sections, with each section including a set of mandrels 320 tacked or laminated thereto. In one embodiment, an adhesive can be used as a processing aid to tack the mandrels to the sheet 328. The sections are separable via appropriately spaced perforations 330. The sheet as shown in FIG. 16 with any number of sections in series can be manufactured, then the sections can be removed so that rotation and bonding of the sheet to itself as described above can take place in order to define a multi-lumen catheter. Such a configuration enables mass-production of the catheter, in one embodiment. Note that the sheets can manufactured in any suitable length so as to form catheter tubes of corresponding length.

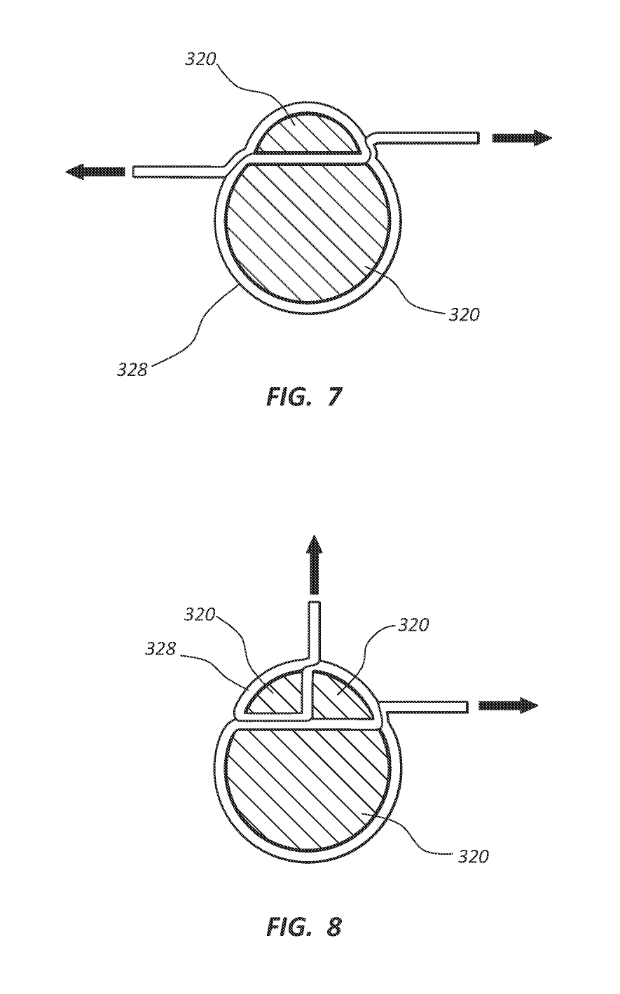

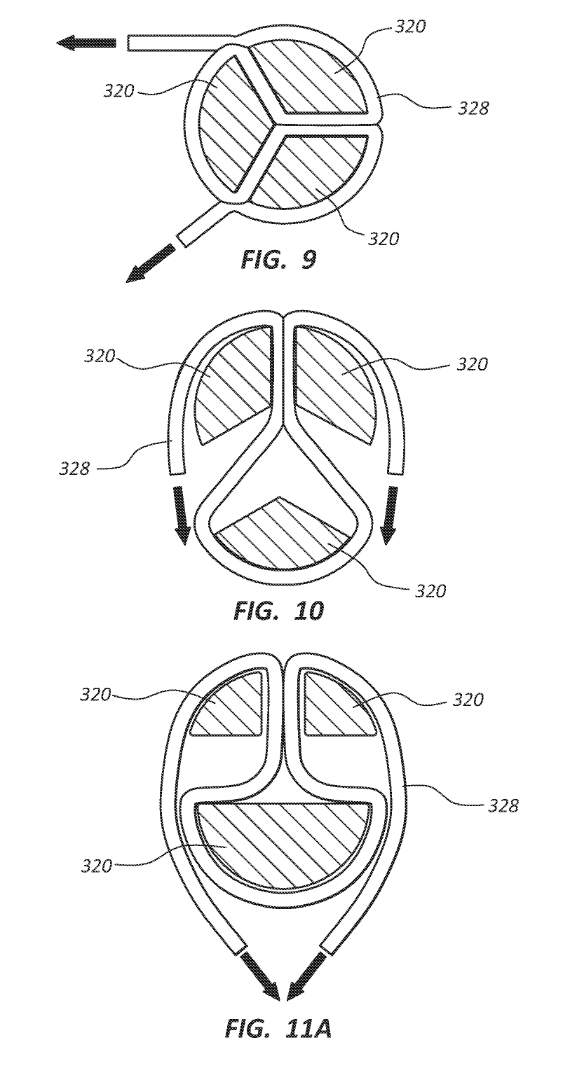

FIGS. 7-10 show other possible sheet wrapping schemes around various shaped mandrels 320 for forming multi-lumen catheter tubes with the forming sheet 328 in a manner similar to that just described above. In particular, FIG. 7 shows a scheme for wrapping the forming sheet 328 around two cooperatively shaped mandrels 320 to define a dual lumen catheter tube, while FIG. 8 shows a three-mandrel configuration for defining a triple lumen catheter tube. FIG. 9 shows one possible sheet wrapping scheme about three mandrels 320 to define a triple lumen catheter tube, while FIG. 10 shows another possible wrapping scheme about the same mandrels as FIG. 9. In these and the other wrapping schemes discussed herein, it is appreciated that weaving of the forming sheet 328 through the various mandrels 320, as opposed to mere mandrel rotation, may be necessary.

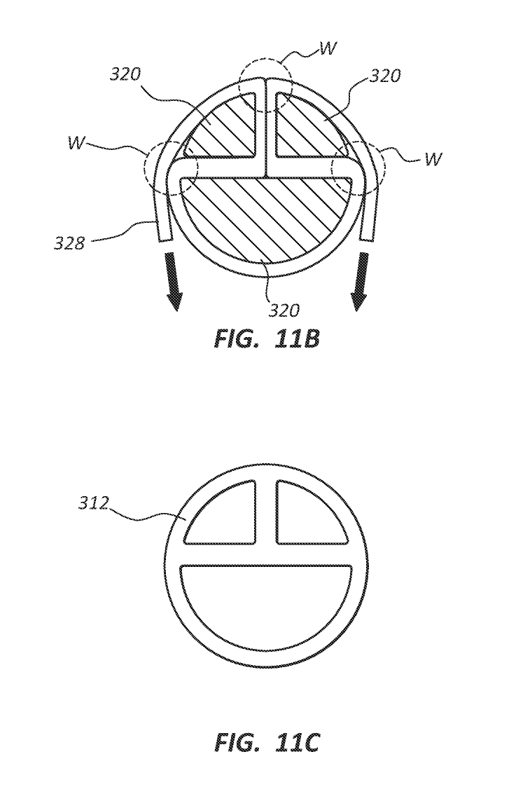

FIGS. 11A-11C show yet another sheet wrapping scheme for producing a triple lumen catheter by wrapping the forming sheet 328 around shaped mandrels 320 before longitudinally trimming and adhering the sheet ends, such as via welding at welding points indicated by “W” in FIG. 11B, to form the catheter tube 312 shown in FIG. 11C. It should therefore be appreciated that a variety of catheter tube configurations can be achieved via wrapping of a flexible sheet of catheter tube material about various mandrel configurations in accordance with the teachings of the present embodiment.

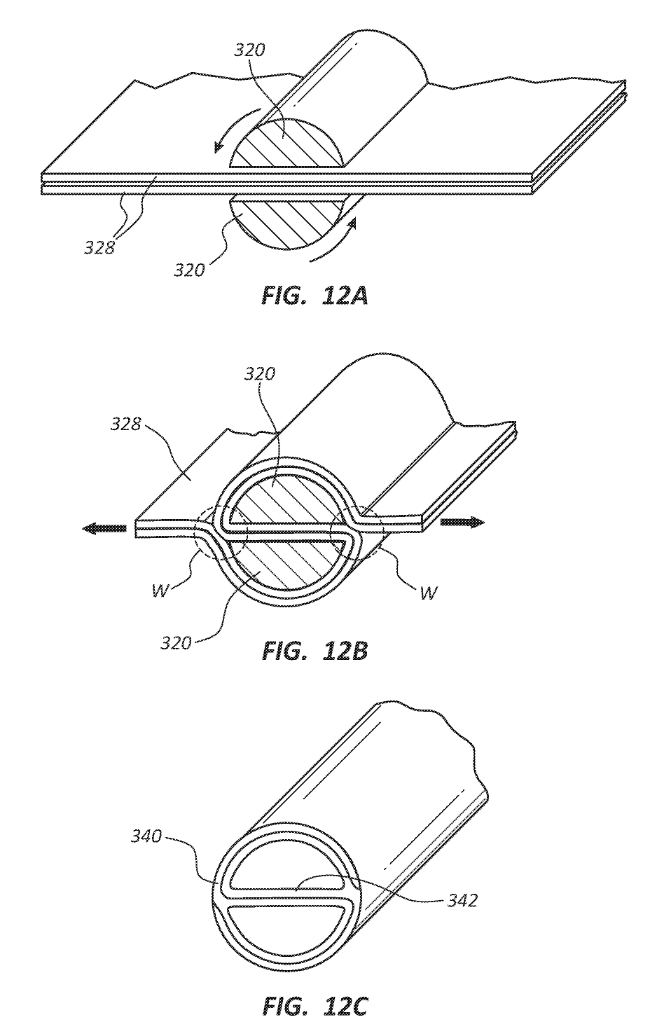

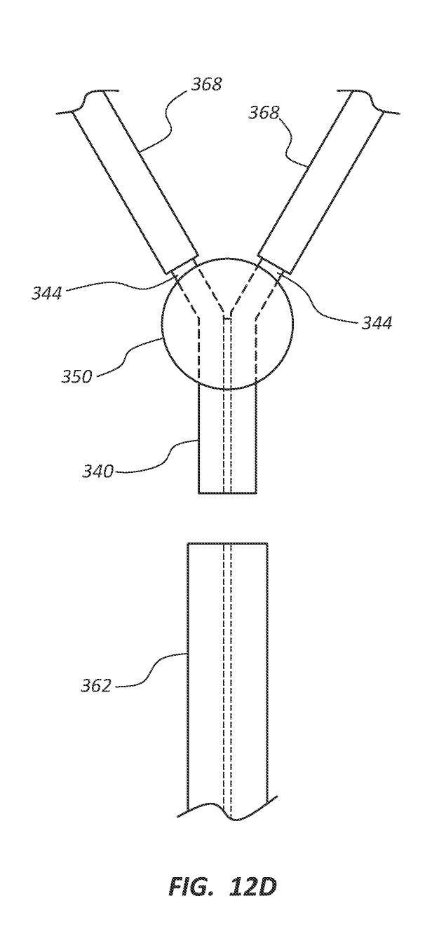

FIGS. 12A-12D depict one embodiment of a method for forming a multi-lumen cannula assembly for use in providing fluid communication between the lumens of a catheter tube and corresponding extension legs of a catheter (see, e.g., FIG. 1). The cannula assembly in one embodiment is included within a bifurcation of a catheter, such as the bifurcation 16 shown in FIG. 1. In particular, FIGS. 12A-12D show that the cannula assembly is manufactured by first interposing two forming sheets 328 of metal or other suitable substance between a pair of D-shaped mandrels 320. The two forming sheets 328 are layered atop one another between the two mandrels 320 (FIG. 12A), after which the mandrels are rotated to wrap the forming sheets about the mandrels, as shown in FIG. 12B. After trimming and joining the remaining sheet ends, such as via welding at welding points indicated at “W” in FIG. 12B, a dual lumen cannula 340 including a septum 342 is formed (FIG. 12C).

The adjacent portions of the forming sheets 328 that define the septum 342 of the cannula 340 are then separated so as to define two independent and diverging cannula lumens 344, shown in FIG. 12D. A bifurcation 350 can be overmolded or otherwise defined on the cannula 340, and the independent cannula lumens 344 can be operably connected with extension legs 368. The cannula 340 of the bifurcation 350 can be operably connected to a multi-lumen catheter 362 to complete the catheter assembly.

In the above-described embodiments utilizing forming sheets, it is appreciated that fewer or more than two forming sheets can be employed. Indeed, in a multi-sheet configuration, each of a plurality of forming sheets can include materials or characteristics distinct from or complementary to the other sheets. For instance, one or more of the sheets used in the above embodiments can include reinforcement structure for reinforcing the final catheter tube to be formed therewith. It is further appreciated that the methods described herein can be employed to manufacture luminal devices of a variety of types and intended purposes.

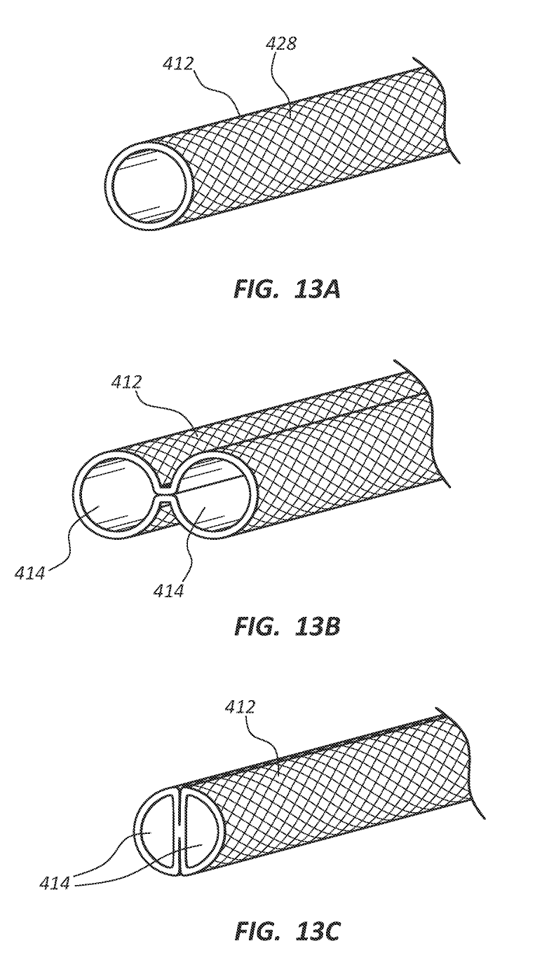

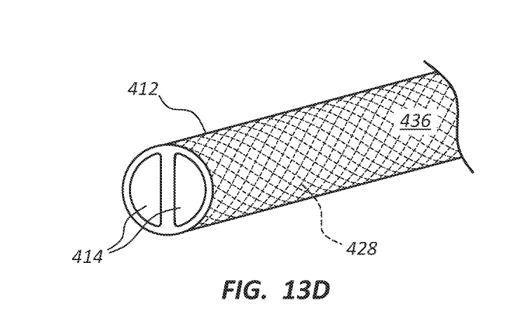

FIGS. 13A-13D depict yet another catheter forming method according to one embodiment, wherein a catheter tube 412 is first formed, via an extrusion or other suitable process. In the present embodiment and as shown in FIG. 13A, the catheter tube 412 is initially a single lumen tube and includes reinforcement 428, such as braiding, for example. It is appreciated that the inclusion of reinforcement in a suitably precise manner can be achieved relatively easier with a single lumen catheter tube than with a multi-lumen catheter tube. As such, the addition of reinforcement to the catheter tube 412 at this stage represents one example of a relatively simple catheter tube reinforcement.

A central portion of the catheter tube 412 is then collapsed longitudinally and opposing surfaces of the collapsed portion are permanently tacked or bonded together along the tube length to subdivide the single lumen of the tube into two lumens 414 (FIG. 13B). In one embodiment a heated die or blade is employed to produce the seam that bonds the collapsed portion to sub-divide the lumens. As seen in FIG. 13B, the cross sectional profile of the catheter tube 412 at this point is substantially a “figure-8” shape.

D-shaped mandrels are then inserted into the lumens 414 to conform each lumen in a D-shaped cross sectional profile (FIG. 13C). Optionally, an outer covering 436 is added to the exterior of the catheter tube 412 and a subsequent heating process is executed to complete manufacture of the catheter tube, as seen in FIG. 13.

It is appreciated that the above process may be used in one embodiment to form from a catheter tube more than two lumens, e.g., three or more lumens defined by longitudinally bonding opposing surfaces of the catheter tube.

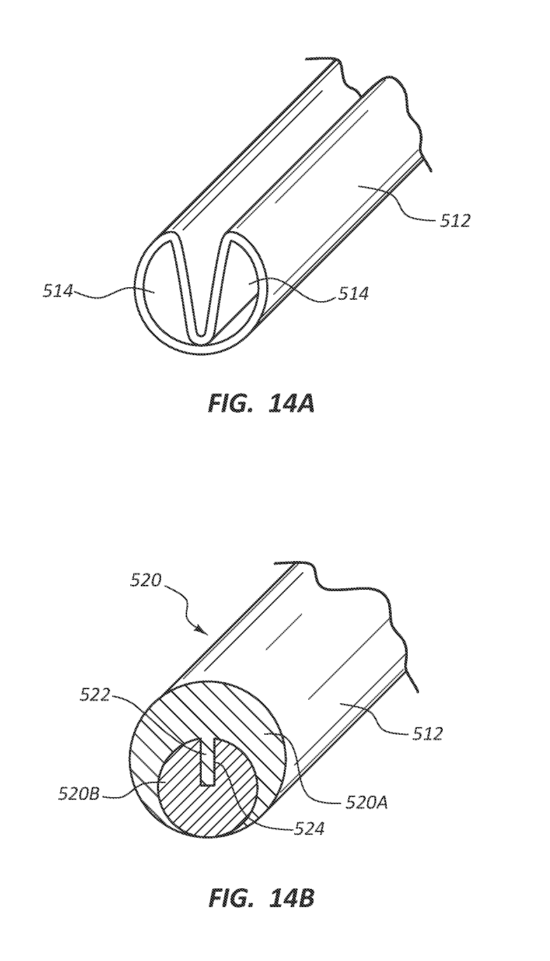

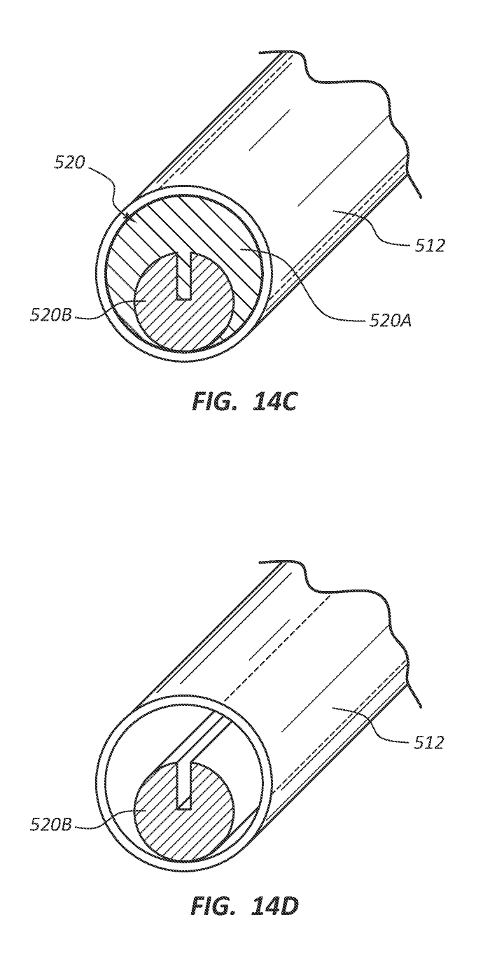

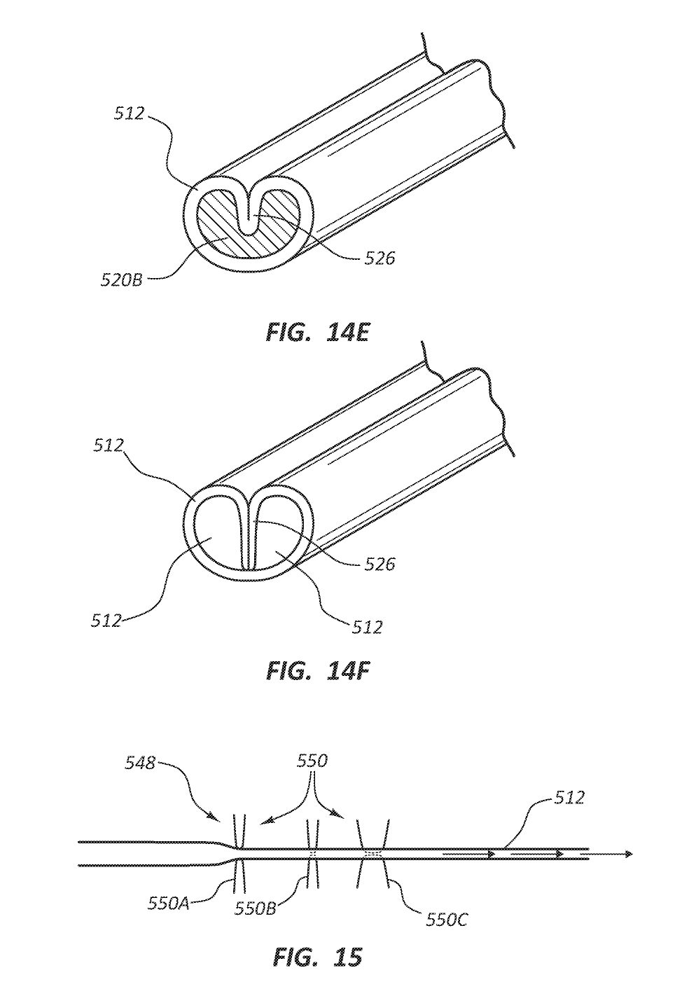

FIGS. 14A-14D depict yet another catheter forming method according to one embodiment, wherein a catheter tube 512 is shown defining two lumens 514 separated by a longitudinally collapsed, V-shaped portion of the outer wall that is bonded to an opposing surface of the catheter wall. FIG. 14A depicts the catheter tube 512 at an intermediate stage of manufacture, shown to illustrate the basic structure of a catheter tube made according to the present embodiment.

In greater detail, the catheter tube 512 is formed in the present embodiment by first forming a single lumen catheter tube, such as that shown in FIG. 14C. A composite beading element (“beading”) 520 shown in FIG. 14B is disposed within the lumen of the catheter tube 512. It is appreciated that the catheter tube 512 can be formed about the beading 520, or the beading can be disposed within the lumen post-tube formation. FIG. 14C shows the beading 520 disposed within the lumen of the catheter tube 512.

As best seen in FIG. 14B, the composite beading 520 includes two separable, interlocking components 520A and 520B, wherein beading component 520A includes a longitudinally extending tab 522 that is received within a slot 524 defined by the beading component 520B when the two beading components are mated together. Note that the particular design and interlocking configuration of the beading components can vary from what is described herein.

In FIG. 14D, beading component 520A is removed from the lumen of the catheter tube 512, leaving only beading component 520B therein. As shown in FIG. 14E, the outer wall of the catheter tube 512 is then collapsed, or folded in on itself by a forming die or other suitable device or method, such that a folded portion 526 of the wall is received into the slot 524 defined in the beading component 520B. The beading component 520B can then be removed while the catheter wall folding, or pinching, continues along the catheter tube length. The pinched, V-shaped folded wall portion 526 of the outer wall is then bonded to the opposing outer wall along the length of the catheter tube to result in the dual lumen catheter tube 512 including two lumens 514, as shown in FIG. 14F. An outer covering, reinforced or not, can be added to the catheter tube 512, if desired. It is also appreciated that the single lumen tube 512 can include reinforcement prior to folding.

It is appreciated that the above process may be used in one embodiment to form from a catheter tube more than two lumens, e.g., three or more lumens defined by longitudinally bonding opposing surfaces of the catheter tube using suitable configured forming elements.

FIG. 15 depicts a method for folding the catheter 512 shown in FIG. 14A, according to another embodiment. In particular, a forming apparatus is employed, which includes various forming elements 550 that cooperate to define dual lumens of the catheter 512. The forming elements 550 are aligned in a row to treat in series a single lumen catheter tube, such as the catheter tube 512 as in FIG. 14C, as the tube is pulled from left to right as shown in FIG. 15. In one embodiment, each forming element includes two knife-like elements that are oppositely disposed one another so as to modify the catheter tube 512 from opposing sides of the catheter wall.

As it is advanced left to right, the catheter tube 512 is first modified by a first forming element 550A, which aligns dual D-shaped beading elements that are disposed within the catheter lumen into proper position. The beading elements are arranged within the single lumen of the catheter tube 512 substantially as shown in FIG. 2, with opposing flat sides of each beading element positioned opposite one another, though other configurations are possible.

After beading element alignment, a forming element 550B folds the catheter tube 512 in on itself so as to assume a centrally collapsed shape such as that shown in FIG. 13B for example, and define a dual lumen structure. The final forming element 550C heats and bonds the centrally collapsed portion of the catheter tube 512 together to provide permanently separate first and second lumens in the catheter tube.

It is appreciated that the forming apparatus described above can be one or more separate devices. Also the forming apparatus so described enables the catheter tube to be aligned, collapsed, and bonded in a continuous process.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

What is claimed is:

1. A method for manufacturing a reinforced multi-lumen catheter tube, the method comprising:
providing a plurality of elongate beading elements;
overextruding a catheter tube over the beading elements, each beading element disposed in and substantially defining a cross sectional profile of a lumen of the catheter tube;
reinforcing the catheter tube; and
extruding an outer covering over the reinforced catheter tube.

2. The method for manufacturing as defined in claim 1, wherein the method further comprises:
removing the beading elements from the reinforced catheter tube after the extrusion of the outer covering.

3. The method for manufacturing as defined in claim 1, wherein reinforcing the catheter tube comprises:
adding a layer of braided reinforcement material about the catheter tube.

4. The method for manufacturing as defined in claim 1, wherein two D-shaped beading elements are employed to define dual lumens of the catheter tube.

5. A method for manufacturing a reinforced multi-lumen catheter tube, the method comprising:
providing at least first and second reinforced single lumen catheter tubes;
inserting a mandrel including a predetermined cross sectional profile into the lumen of at least one of the first and second catheter tubes such that the lumen conforms to the cross sectional profile of the mandrel;
joining the at least first and second catheter tubes with an outer covering to define a multi-lumen catheter tube; and
removing the mandrel from the at least one catheter tube.

6. The method for manufacturing as defined in claim 5, wherein providing each of the first and second reinforced single lumen catheter tubes comprises:
extruding the single lumen catheter tube;
adding a layer of braided reinforcement material about the catheter tube; and
overextruding a cover layer atop the reinforced catheter tube.

7. The method for manufacturing as defined in claim 5, wherein inserting a mandrel further comprises:
inserting a D-shaped mandrel into the lumen of both the first and second catheter tubes such that the lumens of the first and second catheter tubes conform to a D-shaped cross sectional profile.

8. The method for manufacturing as defined in claim 5, wherein joining the at least first and second catheter tubes with the outer covering further comprises:
applying the outer covering over the first and second catheter tubes via a process utilizing heat and pressure.

9. A method for manufacturing a reinforced multi-lumen catheter tube, the method comprising:
extruding at least first and second single lumen catheter tubes over beading elements so as to define a non-round lumen in each catheter tube;
reinforcing at least one of the first and second catheter tubes; and
joining the at least first and second catheter tubes with an outer covering to define a multi-lumen catheter tube.

10. A method for manufacturing a reinforced multi-lumen catheter tube, the method comprising:
providing at least first and second single lumen catheter tubes; and
wrapping a flexible reinforcement sheet about the first and second single lumen catheter tubes to provide a reinforcement layer about each of the first and second catheter tubes.

11. The method for manufacturing as defined in claim 10, further comprising:
covering the at least first and second catheter tubes with an outer covering.

12. The method for manufacturing as defined in claim 10, wherein providing the first and second catheter tubes further comprises:
extruding the first and second single lumen catheter tubes; and
placing a D-shaped mandrel into the lumen of each of the first and the second catheter tubes so that the lumen substantially defines a D-shaped cross sectional profile.

13. The method for manufacturing as defined in claim 10, wherein wrapping the flexible reinforcement sheet about the first and second catheter tubes joins the first catheter tube to the second catheter tube.

14. The method for manufacturing as defined in claim 10, wherein the reinforcement sheet includes a fiber-impregnated thermoplastic or thermoset material.

15. The method for manufacturing as defined in claim 14, wherein the fiber-impregnated includes at least one of metal, nylon, PTFE, and glass.

16. The method for manufacturing as defined in claim 10, wherein wrapping the flexible reinforcement sheet further comprises:
wrapping the flexible reinforcement sheet about the first and second single lumen catheter tubes in an intertwined manner.

17. A method for manufacturing a multi-lumen catheter tube, the method comprising:
providing a single lumen catheter tube; and
joining opposing surfaces of the catheter tube along a longitudinal length thereof to define at least first and second lumens of the catheter tube.

18. The method for manufacturing as defined in claim 17, further comprising:
shaping each of the first and second lumens to a predetermined cross sectional profile.

19. The method for manufacturing as defined in claim 18, wherein shaping the first and second lumens further includes inserting mandrels into the first and second lumens so as to conform the lumens to a cross sectional profile.

20. The method for manufacturing as defined in claim 17, further comprising:
covering the first and second lumens of the catheter tube with an outer covering.

21. The method for manufacturing as defined in claim 17, wherein the single lumen catheter tube is reinforced prior to joining the opposing surfaces, and wherein joining the opposing surfaces of the catheter tube results in a substantially figure-8 shaped cross sectional profile.

22. The method for manufacturing as defined in claim 17, wherein joining the opposing surfaces further comprises:
joining the opposing surfaces of the catheter tube along longitudinal lengths thereof to define first, second, and third lumens of the catheter tube.

23. The method for manufacturing as defined in claim 17, wherein joining the opposing surfaces is accomplished by ultrasonic welding, RF welding, heat staking, or via an adhesive.

24. The method for manufacturing as defined in claim 17, wherein:
providing the single lumen catheter tube further comprises:
providing a beading element within the lumen of the catheter tube, the beading element including longitudinally extending first and second portions, the second portion defining a longitudinal slot along the length thereof, and wherein joining the opposing surfaces of the catheter tube further comprises:
removing the first portion of the beading element from the lumen of the catheter tube;
inserting a folded portion of the catheter tube into the longitudinal slot of the second portion of the beading element along the length of the catheter tube;
removing the second portion of the beading element from the catheter tube; and
joining the folded portion to an opposing surface of the catheter along the longitudinal length thereof to define the first and second lumens.

25. The method for manufacturing as defined in claim 17, wherein:
providing the single lumen catheter tube further comprises:
inserting first and second beading elements within the lumen of the catheter, and wherein joining the opposing surfaces of the catheter tube further comprises:
inserting a folded portion of the catheter tube into a space defined between the first and second beading elements along the longitudinal length of the catheter tube;
removing the first and second beading elements from the catheter tube; and
joining the folded portion to an opposing surface of the catheter tube along the longitudinal length thereof to define the first and second lumens.

26. A method for manufacturing a multi-lumen catheter tube, the method comprising:
clamping a flexible sheet of catheter tube material between first and second mandrels;
rotating the first and second mandrels with respect to the flexible sheet so as to wrap the flexible sheet about the mandrels; and
joining portions of the flexible sheet together along a longitudinal length thereof to define a closed catheter tube including first and second lumens.

27. The method for manufacturing as defined in claim 26, wherein the first and second mandrels each include a predetermined cross sectional profile, and wherein joining portions of the flexible sheet further comprises:
joining portions of the flexible sheet together along the longitudinal length thereof to define the closed catheter tube including first and second lumens that respectively conform to the cross sectional profiles of the first and second mandrels.

28. The method for manufacturing as defined in claim 26, further comprising:
trimming excess flexible sheet material from the catheter tube.

29. The method for manufacturing as defined in claim 26, wherein the flexible sheet includes reinforcement, wherein the catheter tube defines a septum and an outer wall, and wherein the method further comprises
covering the catheter tube with an outer covering.

30. The method for manufacturing as defined in claim 26, wherein a plurality of mandrel sets and flexible sheets are removably arranged in succession on a batch sheet.

31. The method for manufacturing as defined in claim 26, wherein the first and second mandrels are D-shaped, and wherein rotating the mandrels further comprises:
rotating the mandrels approximately 180 degrees about a longitudinal axis such that the flexible sheet substantially conforms to the D-shape of the mandrels.

32. The method for manufacturing as defined in claim 26, wherein the flexible sheet is manufactured via at least one of an extrusion process and a layering process.

33. A method for manufacturing a multi-lumen catheter tube, the method comprising:
providing a plurality of longitudinally aligned mandrels that each define a predetermined cross sectional profile desired for a respective lumen of the catheter tube;
weaving a flexible sheet of catheter tube material about the plurality of aligned mandrels;
joining portions of the sheet together along a longitudinal length thereof to define a closed catheter tube defining a plurality of lumens, each lumen substantially conforming to a cross sectional profile of a respective one of the plurality of mandrels; and
removing the mandrels from the catheter tube.

34. The method for manufacturing as defined in claim 33, wherein joining portions of the sheet includes:
longitudinally welding along a plurality of joints of adjacent portions of the flexible sheet about a circumference of the catheter tube.

35. The method for manufacturing as defined in claim 33, wherein the flexible sheet includes a reinforced catheter tube material to enable the catheter tube to withstand power injection, and wherein the catheter tube includes at least three lumens.

36. A reinforced multi-lumen catheter tube, comprising:
at least first and second single lumen catheter tubes; and
a flexible reinforcement sheet wrapped about the first and second single lumen catheter tubes in an intertwined manner to provide a reinforcement layer about each of the first and second catheter tubes, the reinforcement sheet joining the first catheter tube to the second catheter tube.

37. The reinforced multi-lumen catheter tube as defined in claim 36, further comprising an outer covering applied to the wrapped first and second catheter tubes, the outer covering being applied via a heat and pressure process.
